(19) United States
(12) Patent Application Publication
(10) Pub. No.: US 2012/0122725 A1
(43) Pub. Date: May 17, 2012

(54) Method of determining risk of a neuropsychiatric disorder

(76) Inventors: Stephen Scherer, Toronto (CA); Russell Schachar, Toronto (CA)

(21) Appl. No.: 13/373,149

(22) Filed: Nov. 7, 2011

Related U.S. Application Data
(60) Provisional application No. 61/410,513, filed on Nov. 5, 2010.
(60) Provisional application No. 61/500,256, filed on Jun. 23, 2011.

(51) Int. Cl. C40B 30/04; C12Q 1/68

(52) U.S. Cl. 506/9; 435/6.12; 435/6.11

(57) ABSTRACT

A method of assessing risk of ADHD in a human subject is provided. The method comprises the step of identifying in a nucleic acid-containing sample obtained from the human subject copy number variations associated with one or more genes selected from the group consisting of DCLK1, DCLK2, SORCS3, SORCS1, 16p11.2, ASTN2, MACROD2, CHCHD, CPLX2, ZBBX, PTPRN2 and TRIM32 wherein a determination of copy number variations associated with one or more of said genes is indicative of a risk of ADHD in the human subject.

### FIELD OF THE INVENTION

The present invention relates generally to neuropsychiatric disorders, such as Attention Deficit Hyperactivity Disorder (ADHD) and autism, and more particularly, to methods of assessing in a human subject, the risk of having these conditions using novel biomarkers.

### BACKGROUND OF THE INVENTION

ADHD, characterized by impairing inattention, hyperactivity and impulsiveness, is highly heritable (˜80%), but the underlying genetic determinants are largely unknown. Association studies examining functional candidate genes such as DRD4, SNAP25, DAT1 and DRD5 have yielded some positive findings, but these seem to account for only a small proportion of ADHD variance (˜3%). Moreover, these observations are not yet replicated in genome-wide association studies (GWAS) and linkage scans.

ADHD traits are also commonly observed in other neuropsychiatric conditions like autism spectrum disorder (ASD), schizophrenia and the DiGeorge and Williams-Beuren microdeletion syndromes. Given these observations, and the growing recognition of gene dosage homeostasis in these and other neuropsychiatric disorders, it would be desirable to identify biomarkers linked to one or more of these conditions.

### SUMMARY OF THE INVENTION

Novel biomarkers have now been identified which are indicative of risk of neuropsychiatric disorders such as ADHD, ASD, schizophrenia and the DiGeorge and Williams-Beuren microdeletion syndromes.

Accordingly, in one aspect of the invention, a method of assessing risk in a human subject of a neuropsychiatric disorder is provided, comprising the step of identifying in a nucleic acid-containing sample obtained from the human subject copy number variations associated with one or more genes selected from the group consisting of DCLK1, DKLK2, SORCS3, SORCS1, 16p11.2, ASTN2, MACROD2, CHCHD, CPLX2, ZBBX, PTPRN2 and TRIM32, wherein a determination of copy number variations associated with one or more of said genes is indicative of a risk of ADHD in the human subject.

In another aspect of the invention, a method of assessing risk of ADHD and ASD in a human subject is provided comprising the step of identifying in a nucleic acid-containing sample obtained from the human subject copy number variations associated with the ASTN2 and TRIM32 genes, wherein a determination of said copy number variation is indicative of risk of ADHD and ASD.

These and other aspects of the invention are described herein by reference to the following figures.

### BRIEF DESCRIPTION OF THE FIGURES

FIG. 1 illustrates rare CNVs detected at the 9q33.1 locus overlapping Astrotactin 2 (ASTN2) and/or TRIM32;

FIG. 2 illustrates pedigrees of ADHD families with variants of interest which were absent in controls and a) were de novo or b) occurred at loci previously implicated in ADHD or c) were present in two unrelated ADHD probands or d) overlapped loci previously implicated in other neuro-developmental disorders;

FIG. 3 illustrates pedigrees of four ASD probands with maternally inherited CNVs overlapping Astrotactin 2 (ASTN2) and/or TRIM32;

FIG. 4 illustrates four genomic loci with rare variants in ADHD probands; and

FIG. 5 is a summary of the genotyping method used to identify CNVs in accordance with the present invention.

### DETAILED DESCRIPTION OF THE INVENTION

A method of assessing risk of a neuropsychiatric disorder, e.g. ADHD, ASD, schizophrenia and the DiGeorge and Williams-Beuren microdeletion syndromes, in a human subject is provided. The method comprises identifying in a nucleic acid-containing sample obtained from the human subject copy number variations associated with one or more genes selected from the group consisting of DCLK1, DCLK2, SORCS3, SORCS1, 16p11.2, ASTN2, MACROD2, CHCHD, CPLX2, ZBBX, PTPRN2 and TRIM32. A determination of copy number variations associated with one or more of said neuropsychiatric disorder-related genes is indicative of a risk of a neuropsychiatric disorder in the human subject.

The term “DCLK1” refers to the doublecortin-like kinase 1 gene. This term encompasses the gene sequence NM_{—}001195416 (RefSeq) and functionally equivalent variants thereof. The term “functionally equivalent variant” refers to a gene sequence that may vary from an identified sequence due to the insertions, deletions or substitutions, but which encodes a protein product, e.g. DCLK1 protein, having essentially the same activity.

The term “DCLK2” refers to the doublecortin-like kinase 2 gene. This term encompasses the gene sequence NM_{—}001040260 (RefSeq) and functionally equivalent variants thereof.

The term “SORCS1” refers to the sortilin-related VPS10 domain containing receptor 1 gene. This term encompasses the gene sequence NM_{—}001206570 (RefSeq) and functionally equivalent variants thereof.

The term “SORCS3” refers to the sortilin-related VPS10 domain containing receptor 3 gene. This term encompasses the gene sequence NM_{—}014978 (RefSeq) and functionally equivalent variants thereof.

The term “ASTN2” refers to the astrotactin 2 gene. This term encompasses the gene sequence XM_{—}002346192 (RefSeq) and functionally equivalent variants thereof.

The term “MACROD2” refers to the MACRO domain containing 2 gene. This term encompasses the gene sequence NM_{—}001033087 (RefSeq) and functionally equivalent variants thereof.

The term “CHCHD” refers to the coiled-coil-helix-coiled coil-helix domain containing 3 gene. This term encompasses the gene sequence NM_{—}017812 (RefSeq) and functionally equivalent variants thereof.

The term “CPLX2” refers to the complexin 2 gene. This term encompasses the gene sequence NM_{—}001008220 (RefSeq) and functionally equivalent variants thereof.

The term “ZBBX” refers to the zinc finger, B-box domain containing gene. This term encompasses the gene sequence NM_{—}001199201 (RefSeq) and functionally equivalent variants thereof.

The term “PTPRN2” refers to the protein tyrosine phosphatise, receptor type, N polypeptide 2 gene. This term encompasses the gene sequence NM_{—}002847 (RefSeq) and functionally equivalent variants thereof.

The term “TRIM32” refers to the tripartite motif containing 32 gene. This term encompasses the gene sequence NM_{—}001099679 (RefSeq) and functionally equivalent variants thereof.

The term “16p11.2” refers to a region on the short arm of chromosome 16, within band 11, comprising more than 40 different genes.

All sequences referred to above, e.g. RefSeq sequences, are incorporated herein by reference.

In the present method of determining risk in a human subject of a neuropsychiatric disorder, a biological sample obtained from the subject is utilized. A suitable biological sample may include, for example, a nucleic acid-containing sample or a protein-containing sample. Examples of suitable biological samples include saliva, urine, semen, other bodily fluids or secretions, epithelial cells, cheek cells, hair and the like. Although such non-invasively obtained biological samples are preferred for use in the present method, one of skill in the art will appreciate that invasively-obtained biological samples, may also be used in the method, including for example, blood, serum, bone marrow, cerebrospinal fluid (CSF) and tissue biopsies such as tissue from the cerebellum, spinal cord, prostate, stomach, uterus, small intestine and mammary gland samples. Techniques for the invasive process of obtaining such samples are known to those of skill in the art. The present method may also be utilized in prenatal testing for the risk of ASD using an appropriate biological sample such as amniotic fluid and chorionic villus.

In one aspect, the biological sample is screened for selected genes, e.g. DCLK1, DCLK2, SORCS3, SORCS1, 16p11.2, ASTN2, MACROD2, CHCHD, CPLX2, ZBBX, PTPRN2 and TRIM32, in order to detect mutations in these genes associated with a neuropsychiatric disorder. It may be necessary, or preferable, to extract nucleic acid from the biological sample prior to screening the sample. Methods of nucleic acid extraction are well-known to those of skill in the art and include chemical extraction techniques utilizing phenol-chloroform (Sambrook et al., 1989), guanidine-containing solutions, or CTAB-containing buffers. As well, as a matter of convenience, commercial DNA extraction kits are also widely available from laboratory reagent supply companies, including for example, the QIAamp DNA Blood Minikit available from QIAGEN (Chatsworth, Calif.), or the Extract-N-Amp blood kit available from Sigma (St. Louis, Mo.).

Once an appropriate nucleic acid sample is obtained, it is subjected to well-established methods of screening, such as those described in the specific examples that follow, to detect genetic mutations indicative of a neuropsychiatric disorder, e.g. mutations associated with one or more of the DCLK1, DCLK2, SORCS3, SORCS1, 16p11.2, ASTN2, MACROD2, CHCHD, CPLX2, ZBBX, PTPRN2 and TRIM32 genes. These mutations include genomic copy number variations (CNVs), such as gains and deletions of segments of DNA, for example, gains and deletions of segments of DNA greater than about 10 kb, such as DNA segments greater than 100 kb. Gene mutations or CNVs “associated with” a neuropsychiatric disorder-related gene include CNVs in both coding and regulatory regions of a selected gene. In a preferred embodiment, gene mutations, in the form of CNVs, associated with one or more of the DCLK1, DCLK2, SORCS3, SORCS1, 16p11.2, ASTN2, MACROD2, CHCHD, CPLX2, ZBBX, PTPRN2 and TRIM32 genes, are indicative of a risk of ADHD.

As one of skill in the art will appreciate, gene mutations or CNVs associated with a neuropsychiatric disorder-related gene, e.g. one or more of the DCLK1, DCLK2, SORCS3, SORCS1, 16p11.2, ASTN2, MACROD2, CHCHD, CPLX2, ZBBX, PTPRN2 and TRIM32 genes, are not restricted to a single chromosome, but rather have been detected on multiple chromosomes such as chromosome 4, chromosome 9 and chromosome 10: Examples of CNVs that have been determined to be linked to a neuropsychiatric disorder, e.g. ADHD, include a deletions on chromosome 4, e.g. at 4q31.3, associated with DCLK2, deletions associated with MACROD2, gains on chromosome 10, e.g. at 10q25, associated with SORCS3 and SORCS1, deletions on chromosome 9, e.g. at 9q33.1, associated with ASTN2 and TRIM32, and a deletion associated with the ZBBX gene.

To determine risk of a neuropsychiatric disorder such as ADHD in a human subject, it may be advantageous to screen for multiple CNVs applying array technology. In this regard, genomic sequencing and profiling, using well-established techniques as exemplified herein in the specific examples, may be conducted for a subject to be assessed with respect to ADHD risk/diagnosis using a suitable biological sample obtained from the subject. Identification of one or more CNVs associated with ADHD would be indicative of a risk of ADHD, or may be indicative of a diagnosis of ADHD. This analysis may be conducted in combination with an evaluation of other characteristics of the subject, including for example, phenotypic characteristics.

In view of the determination of gene mutations which are linked to a psychiatric disorder such as ADHD, a method for determining risk of ADHD in a subject is also provided in which the expression or activity of a product of an ADHD-linked gene is determined in a biological protein-containing sample obtained from the subject. Abnormal levels of the gene product or abnormal levels of the activity thereof, i.e. reduced or elevated levels, in comparison with levels that exist in healthy non-ADHD subjects, are indicative of a risk of ADHD, or may be indicative of ADHD. Thus, a determination of the level and/or activity of the gene products of one or more of DCLK1, DCLK2, SORCS3, SORCS1, 16p11.2, ASTN2, MACROD2, CHCHD, CPLX2, ZBBX, PTPRN2 and TRIM32, may be used to determine the risk of ADHD in a subject, or to diagnose ADHD. As one of skill in the art will appreciate, standard assays may be used to identify and quantify the presence and/or activity of a selected gene product.

In another aspect, a method of assessing risk of ADHD in a human subject is provided comprising the step of identifying in a nucleic acid-containing sample obtained from the human subject copy number variations associated with the ASTN2 and TRIM32 genes. A determination of copy number variations associated with ASTN2 and TRIM32 genes is indicative of risk of ADHD. In one embodiment, deletions of at least about 100 kb at 9q33.1 overlapping ASTN2 and TRIM32 is indicative of ADHD.

Related to this aspect, identification of missense mutations in one or both of the ASTN2 and TRIM32 genes may also be indicative of risk of ADHD. In this regard, a missense mutation may result in the expression of a non-functional protein product. Accordingly, in addition to detection of the mutation in a nucleic acid sample of the patient, risk of ADHD and ASD may also be assessed by detection of an altered, e.g. reduced, level and/or activity of the gene products of ASTN2 and TRIM32.

Embodiments of the inventions are described by reference to the following specific example which is not to be construed as limiting.

### Example

### Materials and Methods

### ADHD Probands: Assessment Protocol for Diagnosis

Individuals age 5 to 17 years old (175 boys (72%) and 73 girls (28%)), referred for assessment of attention, learning and behavior problems to The Hospital for Sick Children, Toronto were participants in this study. Participants were included if they met rigorous criteria for ADHD. The assessment entailed a semi-structured diagnostic interview of the participant's parent(s) and of the proband's teacher. The Parent Interview for Child Symptoms (PICS) [Ickowicz, 2006 PMID 16986822] is based on the Schedule for Affective Disorders and Schizophrenia (KSADS) [Ambrosini, 2000 PMID 10638067] with an enhanced module for disruptive behavior disorders. Reliability was assessed through videotaped interviews in 48 cases and was found to be high (interclass correlation for total symptom score=0.93). The Teacher Telephone Interview (TTI; [Tannock, 2002]) is a semi-structured interview conducted by an interviewer with at least a Masters degree in psychology. Reliability of the TTI was assessed using audiotapes and found to be high (interclass correlation for total symptom score=0.93). To receive a diagnosis of ADHD, the participant had to present with impairing and developmentally atypical symptoms before age 7, meet DSM-IV criteria based on PICS and TTI, exhibit evidence of symptoms and impairment both at home and at school, and not present with any of the exclusion criteria for ADHD as stated in DSM-IV (mental retardation, pervasive developmental disorder, or a comorbid psychiatric disorder that could better account for the disorder). Final diagnosis was based on a best-estimate diagnosis arrived at through consensus among the assessing psychiatrist and psychologist. Intelligence and academic attainment were assessed by a clinical psychologist. Parents and children over age 12 years provided consent and younger participants gave assent. The protocol was approved by the Research Ethics Board of the Hospital for Sick Children, Toronto.

### ASD Probands: Assessment Protocol for Diagnosis

The participants in this study were 349 unrelated probands, who met criteria for ASD according to Autism Diagnostic Interview-Revised (ADI-R) and/or Autism Diagnostic Observation Schedule (ADOS) [Risi, 2006 PMID 16926617]. Individuals were recruited from four different Canadian sites: The Hospital for Sick Children, Toronto, Ontario; McMaster University, Hamilton, Ontario; Memorial University, St. John's, Newfoundland and University of Alberta, Edmonton, Alberta.

### Genotyping

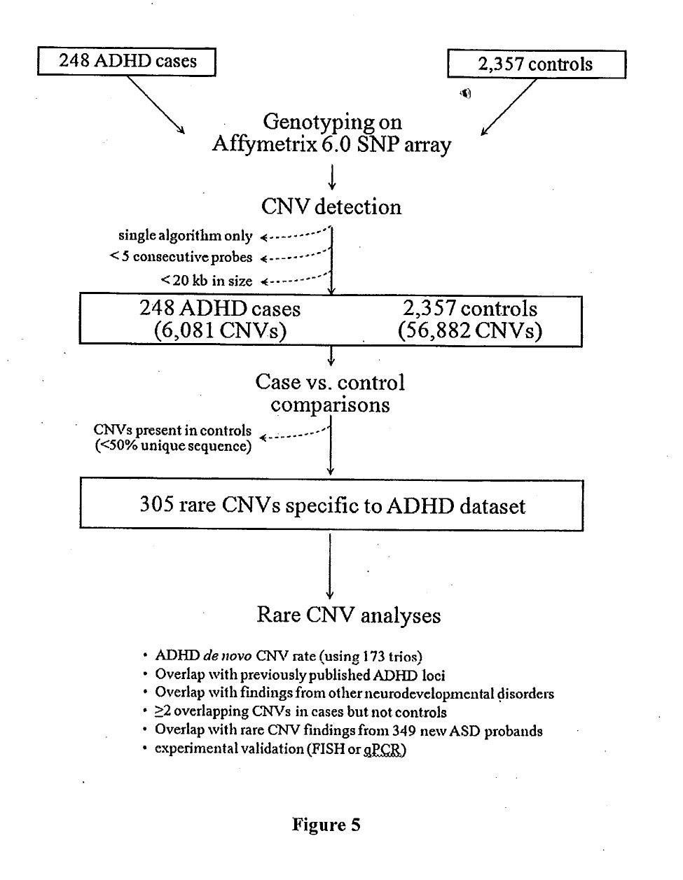

DNA (e.g. from saliva, blood and cell line) collected from ADHD probands and their parents, and from ASD probands, was sent for genotyping at The Centre for Applied Genomics (TCAG) at The Hospital for Sick Children, Toronto. Samples were genotyped on the Affymetrix Genome-Wide Human SNP Array 6.0 using standard protocols as provided by the manufacturer. Arrays meeting Affymetrix quality control guidelines of Contrast QC>0.4 were used for further analysis. A summary of the genotyping methodology used is set out in FIG. 5.

### Population-Based Control Datasets

Two population control datasets, genotyped on the Affymetrix SNP 6.0 array platform and analyzed for CNVs in the same manner as the cases, were used in this study: 1) OHI controls: a cohort of 1,234 control individuals collected as part of a large case control GWAS study at the Ottawa Heart Institute (OM) looking at coronary artery disease in the Ottawa valley [Stewart et al, 2009; PMID: 19371834] and 2) German POPGEN controls—a sample of 1,123 individuals of northern German origin (Schleswig-Holstein) [Krawczak et al, 2006; PMID: 16490960].

Determination of Ancestry from Array Genotypes

In order to control for population stratification and assign accurate ancestries, genotypes from the two case sets and the two control sets were clustered using 1,120 unlinked SNPs by the program STRUCTURE [Pritchard et al, 2000; PMID: 10835412], assuming three ancestral populations. 270 HapMap samples, genotyped on the Affymetrix 6.0 were utilized as references of known ancestry during clustering. Ancestries were assigned using a threshold of co-efficient of ancestry >0.9. Greater than 86% of individuals from the ADHD and ASD case sets and greater than 99% of individuals in both the OHI and POPGEN population control datasets were seen to belong to the European cluster.

### Characterization of Copy Number Variation for Cases and Controls

The array scans were analyzed for copy number variation using three different CNV calling algorithms: Birdsuite [Korn et al, 2008; PMID: 18776909] Affymetrix Genotyping Console (GTC) and iPattern [Zhang et al, Manuscript in preparation]. For each of these algorithms, CNVs spanning 5 or more consecutive array probes and that were at least 20 kb in length were identified. For each sample, overlapping calls from Birdsuite and iPattern were merged using the outside probe boundaries and designated as stringent calls. Singleton calls from Birdsuite or iPattern which overlapped with a GTC call from the same sample were also included in the stringent set. The use of multiple algorithms appeared to reduce the risk of false positive calls algorithms. Samples with number of calls greater than three times the standard deviation from the mean number of calls for an analysis batch were excluded from the study. The case datasets and the control datasets were both analyzed for CNVs in an identical manner as described above. Rare ADHD-specific and ASD-specific variants were defined as those stringent calls in the probands. For the 145 ADHD probands, for whom array data from both parents were available, the PLINK tool set (Purcell et al. Am. J. Hum. Genet. 81, 559-575 (2007)) was used to compute the Mendelian error rate of genotypes from 934, 968 SNP probes on the Affymetrix 6.0 platform. In the 145 trios included in this study, less than 1% of the SNPs exhibited Mendelian errors. Rare CNV results in the ADHD cases were compared with previous published CNV studies in ASD, ADHD, schizophrenia and bipolar disorder, as well as with loci implicated by linkage and association studies of ADHD.

### Experimental Validation of CNV Results

PCR validation of CNV calls was performed in triplicate with SYBRGreen I-based real-time quantitative PCR (qPCR) with controls at the FOXP2 loci at chromosome 7. At least two independent qPCR assays were required for confirmation of a CNV. Using quantitative PCR (qPCR), 33 of the 34 stringent calls that were tested were validated, including all 26 CNVs in Table 1. FISH validation was performed for the de novo gains at the SORCS1/SORCS3 locus.

### Sequencing and Mutation Screening Methods

All coding exons and intron-exon splice sites of TRIM32 and ASTN2 were sequenced in ADHD and ASD cases and in population based controls from the Ontario. Population Genomics Project using standard PCR-based Sanger sequencing. Primer3 software v. 0.4.0 (http://frodo.wi.mit.edu/primer3) was used to design PCR primers. For ASTN2, primers were chosen to cover the longest transcript isoform (NM_{—}198187), which possessed 23 exons as well as the first exon of the shorter isoform (NM_{—}198188). The amplified products were sequenced with the Big Dye Terminator kit using the ABI 3730XL capillary sequencer (Applied Biosystems). Variant detection was performed using SeqScape software from Applied Biosystems. Novel variants detected in the cases, not previously reported in the Single Nucleotide Polymorphism Database (dbSNP) build 130, were validated by resequencing in the case and in samples from both parents, when available.

### Results

### Detection of Structural Variation in ADHD Cases and Population Controls

Using Affymetrix SNP 6.0 microarrays, 248 unrelated ADHD probands (73 female and 175 male) were genotyped at more than 1.8 million markers for CNV interrogation, making this study the highest resolution scan of ADHD genomes to date. In 173 families, where DNA was available from both parents, the complete trio of parents and proband were genotyped. To maximize CNV detection, three different CNV calling algorithms were used as set out above. Subsequent analyses focused on the 10,254 “stringent” CNV calls in the 167 probands, i.e. CNVs found by at least 2 of 3. In this study, all of the 15 stringent calls that were tested were validated by quantitative PCR (qPCR).

In addition, 2,357 population based controls (1,234 from Ontario and 1,123 from Germany—see methods) were analyzed for structural variation using the same array platform and CNV calling strategy as described above. Similar CNV profiles, in terms of number and size of calls, were found in cases and controls.

A dataset of rare, stringently defined CNVs in ADHD probands that were at least 20 kb in length which spanned a minimum of five array-probes, had support from two or more calling algorithms and were not found in controls (less than 50% overlap by length) was defined. Based on these criteria a total of 305 rare CNVs were detected in the ADHD probands. To define ADHD candidate loci, de novo CNVs and rare inherited CNVs which overlapped genetic loci previously implicated in ADHD or in other neuro-psychiatric disorders were prioritized (Table 1).

TABLE 1
De novo and rare inherited CNVs in ADHD probands, which overlapped loci of interest

| | | | | | | | | |
|---|---|---|---|---|---|---|---|---|
| Sample | Sex | CNV | Cytoband | Size (kb) | Inheritance | Ancestry | Genes | Previous reports¹ |

A. Rare CNVs at loci previously implicated in ADHD

| | | | | | | | | |
|---|---|---|---|---|---|---|---|---|
| 9009.3 | M | Gain | 4p16.1 | 482 | P | AS | DRD5, WDR1, SLC2A9 | ADHD |
| 9020.3 | M | Gain | 5q35.2 | 131 | M | EA | CPLX2 | ADHD, SZ |
| 213.3 | F | Gain | 7q32.3 | 159 | U | EA | CHCHD3 | ADHD |
| 6632.3 | M | Gain | 7q36.3 | 1,023 | M | EA | PTPRN2, WDR60, NCAPG2, ESYT2 | ADHD, SZ |
| 6643.3 | F | Gain | 11q13.4 | 147 | U | MIX | 5 genes | ADHD |
| 6584.3 | F | Gain | 15q13 | 3,666 | M | AS | 15q13 locus (16 genes) | ADHD, ASD, SZ |
| 6613.3 | M | Gain | 16p11.2 | 674 | P | EA | 16p11.2 locus (40 genes) | ADHD, ASD, SZ |

B. Overlapping rare CNVs in unrelated ADHD probands

| | | | | | | | | |
|---|---|---|---|---|---|---|---|---|
| 7504.3 | F | Loss | 3q26.1 | 165 | P | EA | ZBBX | ADHD |
| 6455.3 | F | Loss | 3q26.1 | 22 | U | EA | ZBBX | ADHD |
| 191.3 | M | Gain | 6p24.2 | 262 | M | EA | TFAP2A, GCNT2, C6orf218 | ASD |
| 6566.4 | F | Loss | 6p24.2 | 92 | M | EA | GCNT2 | ASD |
| 6587.3 | M | Loss | 9q33.1 | 177 | P | EA | ASTN2, TRIM32 | ASD, BPD, SZ |
| 6604.3 | M | Loss | 9q33.1 | 148 | U³ | EA | ASTN2, TRIM32 | ASD, BPD, SZ |
| 370.3 | M | Gain | 14q23.2 | 293 | P | EA | KCNH5², RHOJ, GPHB5 | ASD, SZ |
| 9056.3 | F | Gain | 14q23.2 | 293 | M | EA | KCNH5², RHOJ, GPHB5 | ASD, SZ |
| 361.3 | M | Loss | 20p12.1 | 542 | M | EA | MACROD2, FLRT3 | ASD |

C. Rare CNVs at loci implicated in other neuro-developmental disorders

| | | | | | | | | |
|---|---|---|---|---|---|---|---|---|
| 292.3 | F | Loss | 4p16 | 97 | M | EA | STK32B | mild MR |
| 9016.3 | M | Loss | 4p12 | 64 | M | EA | GABRG1 | ASD |
| 419.3 | M | Loss | 11q22.1 | 49 | P | EA | CNTN5²(intronic) | ADHD, ASD, SZ |
| 299.3 | M | Gain | 12q24.33 | 211 | P | EA | ANKLE2, POLE, PGAM5, PXMP2 | |
| 6571.3 | M | Gain | 18p11.22 | 368 | M | EA | ANKRD12², RALBP1, TWSG1 | ASD |
| 9025.3 | M | Loss | Xp22.11 | 388 | M | EA | DDX53, upstream of PTCHD1 | ASD, MR |

D. Loci implicated by de novo CNVs in ADHD probands

| | | | | | | | | |
|---|---|---|---|---|---|---|---|---|
| 9232.3 | M | Loss | 4q31.3 | 33 | D | EA | DCLK2 | Epilepsy |
| 102.3 | M | Gain | 10q25.1 | 242 | D | EA | SORCS3 | BPD, ASD |
| | | Gain | 10q25.1 | 318 | D | | SORCS1 | BPD |
| 378.3 | M | Loss | 20p12.1 | 109 | D | EA | MACROD2 | ASD |

Inheritance - P: Paternal; M: Maternal; U: Unknown.
Ancestry - EA: European; AS: East Asian; MIX: Admixture
Genes - Rare CNVs were found at the loci in bold in both the ADHD and the ASD datasets.
¹This column lists the neuropsychiatric disorder(s) in connection to which each locus has been reported by previous studies - references are in the supplementary methods. ADHD: Attention deficit hyperactivity disorder, ASD: Autism spectrum disorder. BPD: Bipolar disorder, MR: Mental retardation, SZ: Schizophrenia.
²Paralogs of these genes have been implicated by previous studies in the neuropsychiatric disorders listed in the last column.
³DNA was not available from the father for testing.

After comparison of the stringent CNVs from cases and controls, rare stringent variants in the ADHD dataset were defined, i.e. those spanning a minimum of 5 array probes and 10 kb in length and possessing at least 50% of total length, as unique, i.e. not present in any of the controls. In order to distinguish potential ADHD risk factors among these rare CNVs, attention was focused on de novo variants and inherited CNVs which overlapped genetic loci previously implicated in ADHD or in other neuro-psychiatric disorders. To further address the issue of potential genetic overlap between ADHD and ASD, rare CNV findings in the ADHD sample set were compared with those from a collection of 349 unrelated ASD probands (see Table 2), assayed for CNVs using an identical array platform and analysis strategy as described for the ADHD cases and population controls.

TABLE 2
Rare CNVs in ASD probands, which overlapped loci of interest

| | | | | | | | | |
|---|---|---|---|---|---|---|---|---|
| | | | | Size | | | | |
| Sample | Sex | CNV | Cytoband | (kb) | Inheritance | Ancestry | Genes | Previous reports¹ |

A. Rare CNVs at loci previously implicated in ASD

| | | | | | | | | |
|---|---|---|---|---|---|---|---|---|
| NA0246 | F | Loss | 3p25.3 | 422 | U | EA | OXTR and 5 other genes | ASD |
| SK0394 | F | Loss | 6q26 | 318 | U | MIX | PACRG, PARK2 | ADHD, ASD, BPD, SZ |
| MM1125 | M | Loss | 11q23.2 | 32 | U | EA | ANKK1, DRD2 | ADHD, ASD, BPD, SZ |
| SK0341 | M | Loss | 14q24.3 | 62 | M | EA | NRXN3² | ASD, SZ, BPD |
| NA0269³ | M | Loss | 16p11.2 | 610 | M | EA | 16p11.2 locus (28 genes) | ADHD, ASD, SZ |
| MM01197 | M | Gain | 16q23.1 | 803 | M | EA | CNTNAP4² | ADHD, ASD, SZ |
| SK0400⁴ | M | Loss | 20p12.1 | 649 | M | EA | MACROD2 | ASD |
| SK0277 | M | Loss | Xp22.11 | 125 | M | EA | DDX53, upstream of PTCHD1 | ASD, MR |

B. Overlapping rare CNVs in unrelated ASD probands

| | | | | | | | | |
|---|---|---|---|---|---|---|---|---|
| MM0308 | F | Loss | 2q37.3 | 4,637 | U | EA | 58 genes | ASD, MR |
| MM0221 | M | Loss | 2q37.3 | 2,734 | U | EA | 39 genes | ASD, MR |
| SK0467 | M | Gain | 9q33.1 | 45 | M | EA | ASTN2 | ASD, BPD, SZ |
| MM1317 | M | Loss | 9q33.1 | 116 | M | EA | ASTN2, TRIM32 | ASD, BPD, SZ |
| NA0066 | M | Loss | 9q33.1 | 93 | M | EA | ASTN2, TRIM32 | ASD, BPD, SZ |
| NA0166 | M | Loss | 9q33.1 | 37 | M | EA | ASTN2 (intronic) | ASD, BPD, SZ |

C. Rare CNVs at loci previously implicated in other neuro-developmental disorders

| | | | | | | | | |
|---|---|---|---|---|---|---|---|---|
| SK0406 | F | Loss | 5q34 | 27 | U | EA | GABRB2 | SZ, BPD |
| SK0353 | M | Gain | 7q32.3 | 114 | M | MIX | CHCHD3 | ADHD |
| MM1296 | M | Loss | 12q24.33 | 108 | M | EA | POLE, PGAM5, PXMP2, ANKLE2 | |

Inheritance - P: Paternal; M: Maternal; U: Unknown.
Ancestry - EA: European; AS: East Asian; MIX: Admixture
Genes - Rare CNVs were found at the loci in bold in both the ADHD and the ASD datasets.
¹This column lists the neuropsychiatric disorder(s) in connection to which each locus has been reported by previous studies - references are in the supplementary methods. ADHD: Attention deficit hyperactivity disorder, ASD: Autism spectrum disorder, BPD: Bipolar disorder, MR: Mental retardation, SZ: Schizophrenia.
²Paralogs of these genes have been implicated by previous studies in the neuropsychiatric disorders listed in the last column.
³Maternally inherited 16p11.2 deletion is also present in autistic brother of the proband. This family was described in Fernandez et al (2009)
⁴Proband was brought in for ASD testing and was found to be dysmorphic with speech apraxia and motor delay. Deletion is absent in autistic brother.

### Loci Implicated by De Novo CNVs in ADHD Cases

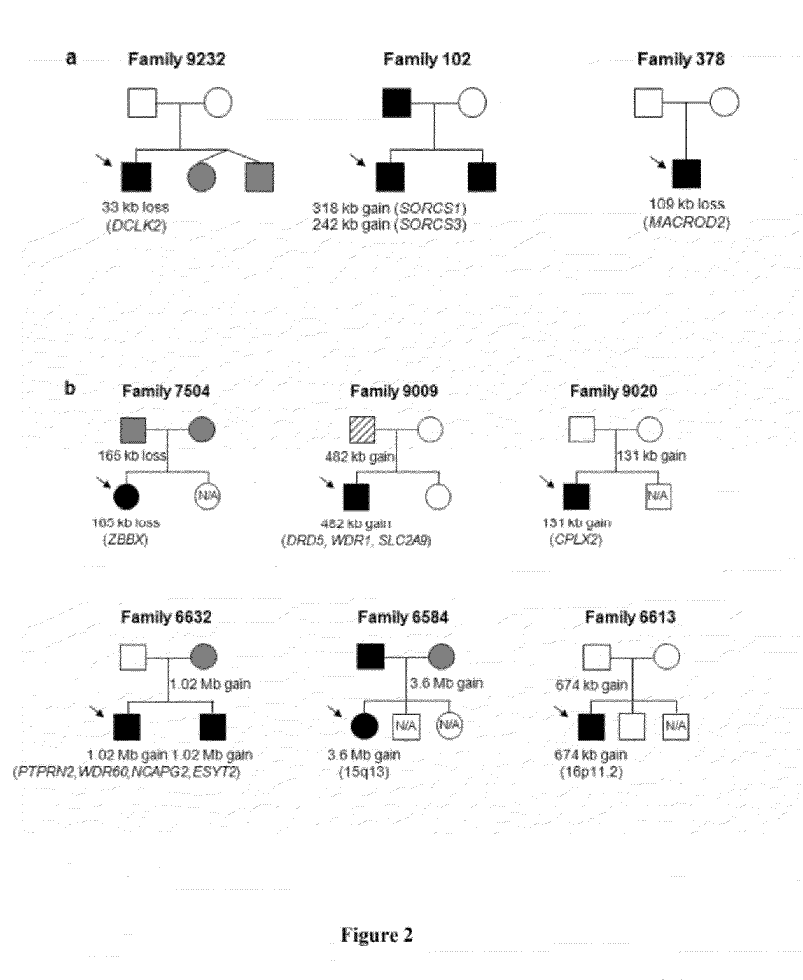

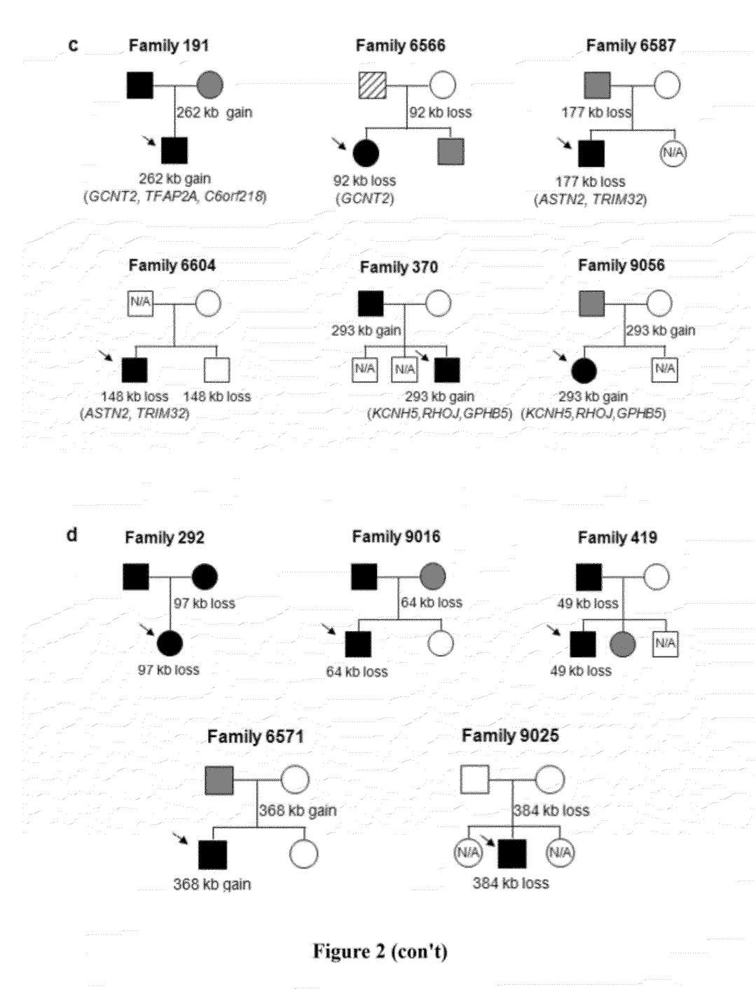

The de novo CNV rate in ADHD from the 145 complete trios after using the PLINK toolset [Purcell, 2007 PMID 17701901] was determined to confirm parentage from the array genotypes of each trio. Detected and experimentally confirmed were de novo variants in 3 probands (2% of trios) as shown in FIG. 2. These included a pair of adjacent gains, of sizes 244 kb and 325 kb at 10q25.1, overlapping the genes SORCS3 and SORCS1, respectively, in a male ADHD proband. SORCS3 and SORCS1 are paralogs encoding VPS10 domain containing receptor proteins, which are involved in intracellular sorting and have been shown to be highly expressed in the developing and mature central nervous system. Upon phenotypic follow-up, it was discovered that the proband, who had met criteria for ADHD at age 8, was later diagnosed with bipolar disorder at age 16. This complex phenotype is particularly intriguing, given the implication of SORCS2 (a paralog of SORCS3 and SORCS1, which maps to chromosome 4) by GWAS studies as a potential risk factor for both ADHD [Lesch, 2008 PMID 18839057] and bipolar disorder [Baum, 2008 PMID 17486107; Ollila, 2009 PMID 19308021]. Since these de novo gains are absent in the probands father and brother, both of whom have a diagnosis for ADHD but not bipolar disorder, it is likely that they contribute to the bipolar disorder phenotype of the proband. An exonic duplication at the SORCS3 locus was reported in an ASD proband, but not in controls in a CNV scan of ASD. In FIG. 2, circles and squares denote females and males respectively while arrows highlight the index proband in each family. Black filled shapes indicate ADHD diagnosis in the individual according to the criteria outlined in the Methods section, unfilled symbols signify unaffected family members, gray shading symbolizes individuals with non-ADHD neuropsychiatric conditions and striped symbols represent individuals with some ADHD traits, but no definitive ADHD diagnosis. N/A denotes individuals, from whom no DNA was available for testing.

The second de novo variant detected was a 33 kb de novo deletion overlapping 2 exons of the DCLK2 (doublecortin-like kinase 2) gene at 4q31.3, in a male proband with combined type ADHD and anxiety traits, who also exhibited seizure symptoms. DCLK2 is a recently characterized gene expressed both in proliferating cells and post-mitotic neurons, consistent with the known roles of its paralogs DCLK1 and DCX in neuronal migration. Mutations in DCX have been associated with epilepsy and periodic limb movements, and the mouse double-knockout model for DCX and DCLK2 exhibited altered hippocampal maturation and spontaneous seizures [Keijan, 2009 PMID 19342486]. The DCLK1 region was one of the top findings in a recent GWAS study of 958 ADHD probands [Neale, 2008 PMID 18980221].

Another de novo deletion was exonic to the MACROD2 gene at 20p12.1, in a male ADHD proband. Although not much is known about the function of the MACROD2 gene, other than potential imprinting and expression in the brain [Luedi, 2007 PMID 18055845]. Further support for the pathogenicity of this locus comes from the presence of a rare, maternally inherited deletion exonic to MACROD2 and FLRT3, a neuronal cell adhesion gene intrinsic to MACROD2, in another ADHD proband in the study. An exonic de novo deletion was also detected at the MACROD2 locus in one of the ASD probands from the AGP study [Pinto AGP, 2010 submitted].

### Rare CNVs at Loci Previously Implicated in ADHD

In addition to the afore-mentioned de novo variants, several rare inherited CNVs were detected in ADHD probands but not in controls, which overlapped loci previously reported in genetic studies of ADHD. These included large inherited gains of sizes 675 kb and 3.3 Mb at the 16p11.2 and 15q13 loci in two ADHD probands, in keeping with reports of ADHD being a frequent phenotypic component of these micro-duplication/deletion syndromes [Shinawi, 2009 PMID 19914906; Miller, 2009 PMID 18805830]. Other rare duplications of interest at loci previously associated with ADHD were detected at 4p16.1 and 7q36.3, in two male ADHD probands overlapping DRD5 and PTPRN2 respectively, among other genes. The dopamine receptor subtype D5 gene (DRD5), encompassed by a duplication transmitted from an affected father, has strong evidence for association to ADHD from multiple studies [Daly, 1999 PMID 10208453; Barr, 2000 PMID 11032390; Faraone, 2006 PMID 16961425]. The duplication at the 7q36.3 locus, detected in two brothers with ADHD, overlaps four genes including PTPRN2, which encodes a member of the protein tyrosine phosphatase (PTP) family of signaling molecules. This genetic locus has been reported earlier in connection with quantitative ADHD phenotypes by a family based association study of IMAGE/GAIN data [Lasky-Su, 2008 PMID 18821565]. Deletion of the mouse homolog of this gene, IA-2β has been recently reported to cause behavioral and learning disturbances via a decrease in the global levels of neurotransmitter release [Nishimura, 2009 PMID 19361477].

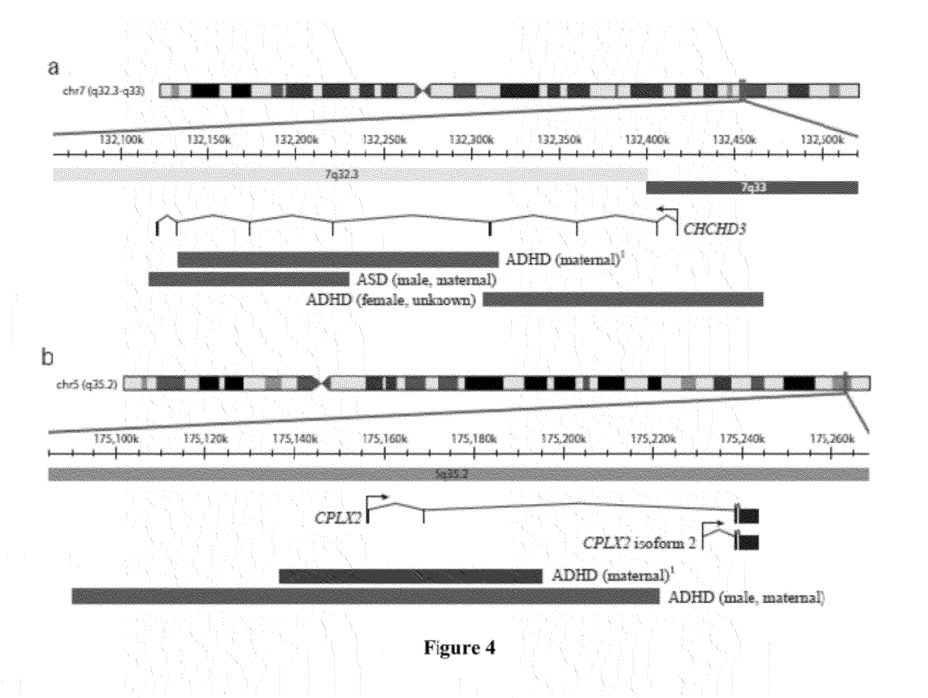

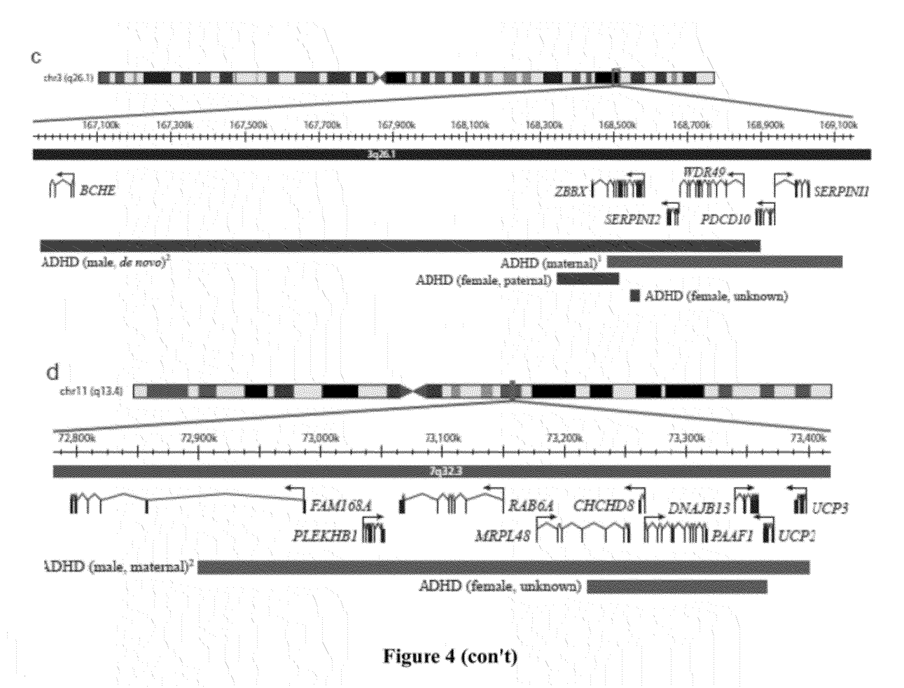

On comparison of the present results with those loci reported by two recently published studies of rare copy number variation in ADHD [Elia, 2009 PMID 19546859; Lesch, 2010 PMID 20308990], there were four regions of overlap at 3q26.1 (ZBBX), 5q35.2 (CPLX2), 7q32.3 (CHCHD3) and 11q13.4 at the 3q26.1 region, implicated by a large de novo deletion in a male ADHD proband by Lesch et al [Lesch, 2010 PMID 20308990], smaller deletions exonic to the ZBBX gene, in two unrelated ADHD probands, were detected (FIG. 4). This gene, which encodes a zinc finger domain protein of unknown function, was also disrupted by a duplication CNV in an ADHD proband in the study by Elia et al. [Elia, 2009 PMID 19546859]. The Complexin 2 (CPLX2) finding is particularly intriguing given its co-localization with the SNAP-25 protein in the SNARE complex, as part of its role in regulating neurotransmitter release during synaptic vesicle fusion. The SNAP25 gene has been implicated in ADHD via a candidate gene association approach previously. Cplx2 knockout mice were viable but exhibited behavioral deficits and cognitive abnormalities. The genes overlapped by the variants at the two other regions of overlap included CHCHD3 and CHCHD8, which belong to the coiled-coil-helix domain-containing gene family.

### Rare CNVs at Loci Previously Implicated in Other Neurodevelopmental Disorders

Overlap of rare CNVs in the ADHD cohort with those previously implicated in other neuropsychiatric disorders, notably ASD, including deletions at genes CNTN5, GABRG1, GCNT2 and STK32B were also observed.

### Overlap Between Rare CNV Findings in ADHD and ASD

To identify rare CNVs in the ADHD dataset that may also be linked to ASD, the case dataset was expanded to include 349 newly characterized ASD probands. ASD was selected as a first comparator due to the high rate of co-occurrence of ADHD in ASD. In order to standardize the initial data and allow the most robust comparison, the same microarray platform and CNV calling strategy for the ASD, ADHD cohort and control cohorts was used. Genetic loci highlighted by the occurrence of rare CNVs in both the ASD and ADHD datasets included those previously mentioned (16p11.2 locus, MACROD2 and CHCHD3) as well as the 12q24.33 region and the X-linked DDX53/PTCHD1 locus (Table 3).

TABLE 3
Rare CNVs in ASD probands, overlap loci also implicated in ADHD probands

| | | | Start | End | | | | | |
|---|---|---|---|---|---|---|---|---|---|
| Sample | Sex | Chr | (Build36/hg18) | (Build36/hg18) | Size(bp) | CNV | Inh | Ancestry | Genes |
| 88032 | M | 7 | 132,116,272 | 132,230,166 | 113,895 | Gain | M | MIX | CHCHD3 |
| 136929 | M | 9 | 118,263,609 | 118,308,641 | 45,033 | Gain | M | EA | ASTN2 |
| 128963 | M | 9 | 118,407,129 | 118,523,510 | 116,382 | Loss | M | EA | ASTN2, TRIM32 |
| 64119 | M | 9 | 118,480,042 | 118,570,447 | 90,406 | Loss | M | EA | ASTN2, TRIM32 |
| 84657 | M | 9 | 118,568,208 | 118,605,377 | 37,170 | Loss | M | EA | ASTN2 (intronic) |
| 124498 | M | 12 | 131,714,545 | 131,822,519 | 107,975 | Loss | D | EA | POLE, PGAM5, PXMP2, ANKLE2 |
| 121851¹ | M | 16 | 29,474,810 | 30,085,308 | 610,499 | Loss | M | EA | 16p11.2 locus (28 genes) |
| 109909² | M | 20 | 14,887,620 | 15,537,065 | 649,446 | Loss | M | EA | MACROD2 |
| 91548 | M | X | 22,891,502 | 23,015,874 | 124,373 | Loss | M | EA | DDX53, upstream of PTCHD1 |

All variants in table above were validated by qPCR
Inh (Inheritance) - M: Maternal; D: De Novo.
Ancestry - EA: European; MIX: Admixture.
Genes - Rare CNVs were found at the loci in bold in both the ADHD and the ASD datasets.
¹Maternally inherited 16p11.2 deletion is also present in autistic brother of the proband. This family was described in Fernandez et al (2009)
²Proband was brought in for ASD testing and was found to be dysmorphic with speech apraxia and motor delay. Deletion is absent in autistic brother.

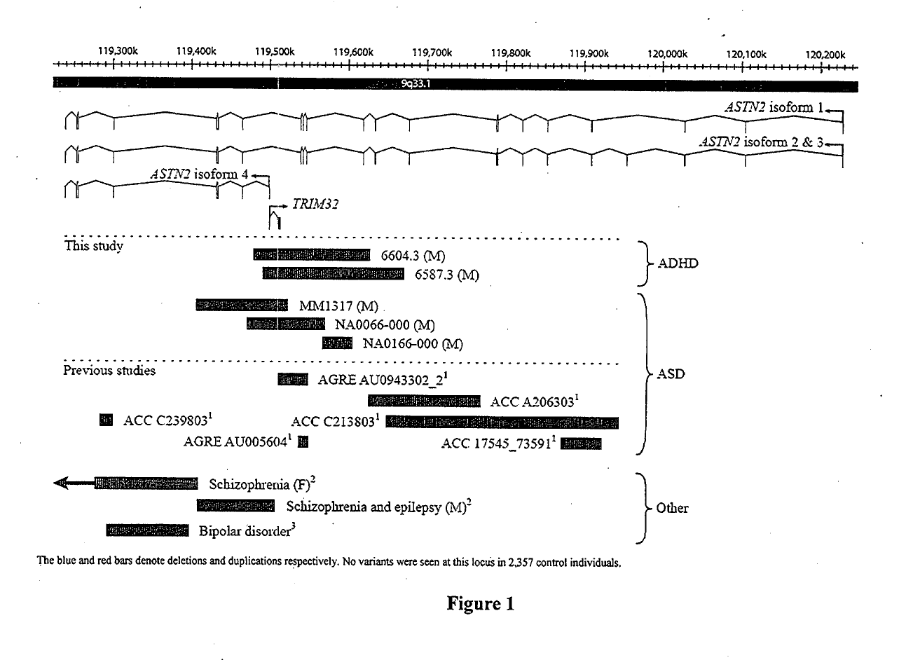

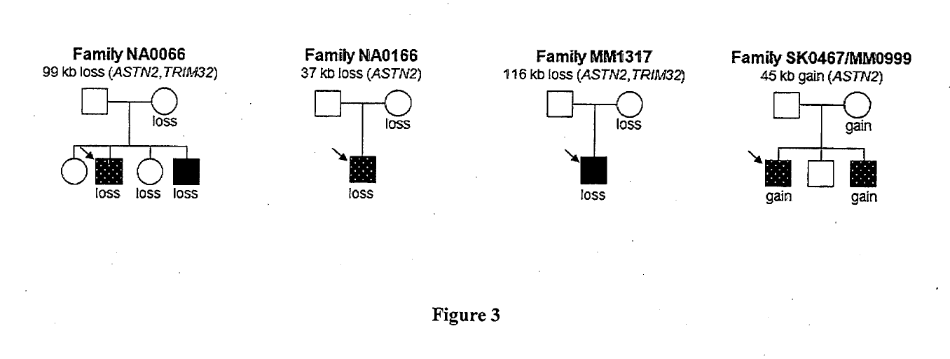

The most intriguing finding was a significant enrichment of rare deletions at 9q33.1 overlapping ASTN2 and TRIM32 in four ASD (˜1%) and two ADHD probands (˜1%) and no controls (Fisher's exact test two tailed p-value=6.68×10⁻⁵) (FIG. 1). ASTN2 encodes astrotactin 2, a neuronal membrane protein exhibiting abundant expression in the cerebellum in both the fetal and adult brain. Three of the ASD probands met criteria for ADHD as measured by the Connor's hyperactivity questionnaire, while the fourth showed signs of hyperactivity, but was below the threshold for ADHD diagnosis as shown in FIG. 3. These findings highlight the potential contribution of this locus to the presence of ADHD symptoms in ASD. While exceptionally rare CNVs at this locus have been reported in earlier CNV studies of ASD, bipolar disorder and schizophrenia, this is the first report of CNVs at this locus in ADHD. In FIG. 3, black filled symbols represent ASD diagnosis while dots symbolize individuals who met criteria for ADHD as measured by the Connor's hyperactivity questionnaire.

### DNA Sequencing of ASTN2 and TRIM32

To further assess if smaller nucleotide-resolution mutations might also be found in ASTN2 and/or TRIM32, DNA sequencing of all coding exons and splice sites in each of these genes in ADHD and ASD cases was conducted, as well as in controls. The five ASTN2/TRIM32 cases from this study carrying deletions at this locus were also sequenced. Non-sense mutations predicted to lead to ASTN2 or TRIM32 haploinsufficiency in a manner similar to CNV were not, detected. Novel missense variants in the ASTN2 gene in 8/276 ADHD cases, 9/346 ASD cases and 2/188 controls (Tables 4 to 7). TRIM32 sequencing revealed missense variants in 4/257 AMID cases, 6/357 ASD cases and 2/157 controls (Tables 8 to 10). Interestingly, one ASD proband was seen to have novel missense variants in both the TRIM32 and ASTN2 genes. Several of the missense variants in the cases (6/8 ADHD and 7/9 ASD) occurred at positions seen to be highly conserved across all vertebrates.

TABLE 4
Summary of ASTN2 and TRIM32 exon sequence variants in ADHD,
ASD and control samples

| | | Individuals | Individuals | | |
|---|---|---|---|---|---|
| | | with | without | | |
| | | missense | missense | | |
| Gene | Dataset | variants | variants | Total | p-value |
| ASTN2 | ADHD | 8 | 268 | 276 | 0.2130 |
| | ASD | 9 | 337 | 346 | 0.3432 |
| | ADHD & ASD | 17 | 605 | 622 | 0.272 |
| | Controls | 2 | 186 | 188 | — |
| TRIM32 | ADHD | 4 | 253 | 257 | 1 |
| | ASD | 6 | 351 | 357 | 1 |
| | ADHD & ASD | 10 | 604 | 614 | 1 |
| | Controls | 2 | 155 | 157 | — |

TABLE 5
Novel ASTN2 sequence variants in ADHD probands

| | | | | Variant | Amino Acid | | | Freq in | Freq in |
|---|---|---|---|---|---|---|---|---|---|
| Sample | Sex | Change | Variant (DNA) | (Protein) | change | Exon | Inheritance | cases | controls |
| 112353 | M | Missense | c160C > A (het) | p20P > Q | Proline to | Exon 1 | Paternal | 1 in 276 | 0 in 188 |
| | | | | (het) | Glutamine | | | | |
| 115361 | M | Missense | c870T > A (het) | p257S > T | Serine to | Exon 3 | Paternal | 3 in 276 | 0 in 378 |
| | | | | (het) | Threonine | | | | |
| 121771 | M | Missense | c870T > A (het) | p257S > T | Serine to | Exon 3 | Paternal | 3 in 276 | 0 in 378 |
| | | | | (het) | Threonine | | | | |
| 121768 | F | Missense | c2187G > A (het) | p696D > N | Aspartate to | Exon 12 | Present in | 1 in 276 | 0 in 378 |
| | | | | (het) | Asparagine | | both | | |
| | | | | | | | parents | | |
| 186-3 | M | Missense | c2121G > A (het) | p674V > M | Valine to | Exon 11 | Maternal | 1 in 276 | 0 in 188 |
| | | | | (het) | Methionine | | | | |
| 141667 | M | Missense | c870T > A (het) | p257S > T | Serine to | Exon 3 | Paternal | 3 in 276 | 0 in 378 |
| | | | | (het) | Threonine | | | | |
| 139948 | M | Missense | c3664C > T(het) | p1188T > M | Threonine to | Exon 22 | Maternal | 1 in 276 | 0 in 188 |
| | | | | (het) | Methionine | | | | |
| 133153 | M | Missense | c2990G > A(het) | P963M > I | Methionine | Exon 18 | Maternal | 1 in 276 | 0 in 188 |
| | | | | (het) | to Isoleucine | | | | |

TABLE 6
Novel ASTN2 sequence variants in ASD probands

| | | | | | Amino Acid | | | Freq in | Freq in |
|---|---|---|---|---|---|---|---|---|---|
| Sample | Sex | Change | Variant (DNA) | Variant (Protein) | change | Exon | Inheritance | cases | controls |
| 60504L | M | Missense | c1963T > G (het) | p.621L > R (het) | Leucine to | Exon 10 | Paternal | 1 in 346 | 0 in 378 |
| | | | | | Arginine | | | | |
| 82359L | M | Missense | c2452G > A (het) | p.784R > Q (het) | Arginine to | Exon 13 | Paternal | 1 in 346 | 0 in 188 |
| | | | | | Glutamine | | | | |
| 109003 | M | Missense | c1179C > T (het) | p.360R > C (het) | Arginine to | Exon 4 | Not maternal | 1 in 346 | 0 in 188 |
| | | | | | Cysteine | | | | |
| 97132L | M | Missense | c3084A > G (het) | p.995K > E (het) | Lysine to | Exon 18 | Maternal | 1 in 346 | 0 in 188 |
| | | | | | Glutamate | | | | |
| 104049L | M | Missense | c1629G > T (het) | p.510V > L (het) | Valine to | Exon 7 | Maternal | 1 in 346 | 0 in 188 |
| | | | | | Leucine | | | | |
| 139374L | M | Missense | c3501A > C (het) | p.1134T > P (het) | Threonine to | Exon 21 | Maternal | 1 in 346 | 0 in 188 |
| | | | | | Proline | | | | |
| 93613 | M | Missense | c3502C > T (het) | p.1134T > I (het) | Threonine to | Exon 21 | Paternal | 1 in 346 | 0 in 188 |
| | | | | | Isoleucine | | | | |
| 122687L | F | Missense | c870T > A (het) | p.257S > T (het) | Serine to | Exon 3 | N/A | 1 in 346 | 0 in 378 |
| | | | | | Threonine | | | | |
| 96279L | M | Missense | c2633G > T (het) | p.844R > S (het) | Arginine to | Exon 15 | N/A | 1 in 346 | 0 in 188 |
| | | | | | Serine | | | | |

TABLE 7
Novel ASTN2 sequence variants in controls

| | | | | | Amino Acid | | | | Freq in |
|---|---|---|---|---|---|---|---|---|---|
| Sample | Sex | Change | Variant (DNA) | Variant (Protein) | change | Exon | Inh. | Freq in cases | controls |
| 88919 | M | Missense | c807C > T (het) | p236R > C (het) | Arginine to | Exon 3 | N/A | 0 in 346 ASD 0 | 1 in 188 |
| | | | | | Cysteine | | | in 276 ADHD | |
| 89967 | F | Missense | c3312C > T(het) | p1071R > W(het) | Arginine to | Exon 20 | N/A | 0 in 346 ASD 0 | 1 in 188 |
| | | | | | Tryptophan | | | in 276 ADHD | |

TABLE 8
Novel TRIM32 sequence variants in ADHD probands

| | | | | | Amino Acid | | | Freq in | Freq in |
|---|---|---|---|---|---|---|---|---|---|
| Sample | Sex | Change | Variant (DNA) | Variant (Protein) | change | Exon | Inheritance | cases | controls |
| 131275 | M | Missense | c1902A > C (het) | p581M > L (het) | Methionine to | Exon 2 | Probably | 1 in 257 | 0 in 157 |
| | | | | | Leucine | | maternal | | |
| 87-3 | M | Missense | c1975G > A (het) | p605G > D (het) | Glycine to | Exon 2 | Maternal | 1 in 257 | 0 in 157 |
| | | | | | Aspartate | | | | |
| 138761 | F | Missense | c1140A > G (het) | p327M > L (het) | Methionine to | Exon 2 | N/A | 1 in 257 | 0 in 157 |
| | | | | | Leucine | | | | |
| 146248 | M | Missense | c933G > A (het) | p258A > T (het) | Alanine to | Exon 2 | N/A | 1 in 257 | 0 in 157 |
| | | | | | Threonine | | | | |

TABLE 9
Novel TRIM32 sequence variants in ASD probands

| | | | | | Amino Acid | | | Freq in | Freq in |
|---|---|---|---|---|---|---|---|---|---|
| Sample | Sex | Change | Variant (DNA) | Variant (Protein) | change | Exon | Inheritance | cases | controls |
| 60504L | M | Missense | c.1099 C > T (het) | p.313 A > V (het) | Alanine to | Exon 2 | Maternal | 1 in 357 | 0 in 157 |
| | | | | | Valine | | | | |
| 58568 | M | Missense | c.1366 G > A (het) | p.402 G > D (het) | Glycine to | Exon 2 | Maternal | 2 in 357 | 0 in 157 |
| | | | | | Aspartate | | | | |
| 96277L | M | Missense | c.1366 G > A (het) | p.402 G > D (het) | Glycine to | Exon 2 | Maternal | 2 in 357 | 0 in 157 |
| | | | | | Aspartate | | | | |
| 105532 | M | Missense | c.1173 G > A (het) | p.338 A > T (het) | Alanine to | Exon 2 | Probably | 1 in 357 | 0 in 157 |
| | | | | | Threonine | | maternal | | |
| 77447 | M | Missense | c.2115A > G (het) | p.652T > A (het) | Threonine to | Exon 2 | Maternal | 1 in 357 | 0 in 157 |
| | | | | | Alanine | | | | |
| 117482 | M | Missense | c.1140A > G(het) | p.327M > V (het) | Methionine to | Exon 2 | Paternal | 1 in 357 | 0 in 157 |
| | | | | | Valine | | | | |

TABLE 10
Novel TRIM32 sequence variants in controls

| | | | | | Amino Acid | | | | Freq in |
|---|---|---|---|---|---|---|---|---|---|
| Sample | Sex | Change | Variant (DNA) | Variant (Protein) | change | Exon | Inh. | Freq in cases | controls |
| 87451 | M | Missense | c.595 G > A (het) | p. 145R > Q (het) | Arginine to | Exon 2 | N/A | 0 in 357 ASD 0 | 1 in 157 |
| | | | | | Glutamine | | | in 257 ADHD | |
| 88874 | M | Missense | c719G > C (het) | p186Q > H (het) | Glutamine to | Exon 2 | N/A | 0 in 357 ASD 0 | 1 in 157 |
| | | | | | Histidine | | | in 257 ADHD | |

We claim:

1. A method of assessing risk in a human subject of ADHD comprising the step of identifying in a nucleic acid-containing sample obtained from the human subject copy number variations associated with one or more genes selected from the group consisting of DCLK1, DCLK2, SORCS3, SORCS1, 16p11.2, ASTN2, MACROD2, CHCHD, CPLX2, ZBBX, PTPRN2 and TRIM32 wherein a determination of copy number variations associated with one or more of said genes is indicative of a risk of ADHD in the human subject.

2. The method of claim 1, wherein the CNV is a gain of greater than 100 kb.

3. The method of claim 2, wherein the CNV gain overlaps SORCS3 or SORCS1.

4. The method of claim 1, wherein the CNV is a deletion.

5. The method of claim 4, wherein the CNV deletion is associated with at least one of DCLK2, MACROD2, ASTN2 and TRIM32.

6. A method of assessing risk of ADHD in a human subject is provided comprising the step of identifying in a nucleic acid-containing sample obtained from the human subject copy number variations associated with the ASTN2 and TRIM32 genes, wherein a determination of said copy number variation is indicative of risk of ADHD.

7. The method of claim 6, wherein the CNVs are deletions of at least about 100 kb associated with ASTN2 and TRIM32.

8. The method of claim 7, wherein the CNV deletion occurs at chromosome 9q33.1 overlapping ASTN2 and TRIM32.
